(19) United States
(12) Patent Application Publication
(10) Pub. No.: US 2014/0343409 A1
(43) Pub. Date: Nov. 20, 2014

(54) INFLATION APPARATUS WITH PRESSURE RELIEF, RELATED SYSTEMS, METHODS AND KITS

(71) Applicant: NinePoint Medical, Inc., Cambridge, MA (US)

(72) Inventors: George J. Purtell, Westford, MA (US); Narissa Y. Chang, Somerville, MA (US); Michael Madden, Princeton, MA (US); Eman Namati, Arlington, MA (US)

(21) Appl. No.: 14/263,354

(22) Filed: Apr. 28, 2014

Related U.S. Application Data
(60) Provisional application No. 61/824,496, filed on May 17, 2013.

(51) Int. Cl. A61B 5/00

(52) U.S. Cl. CPC A61B 5/0084
USPC 600/425

(57) ABSTRACT

An inflation system is provided including a housing having an interior portion including an opening. A connector is positioned within the interior portion. The connector includes a first portion defining a first passageway and a second portion defining a second passageway. The second portion includes a pressure relief valve. A shaft defines a third passageway. The shaft extends through the first passageway. An inflatable member defines a fourth passageway. The inflatable member includes a first end coupled to the connector and a second end positioned external to the interior portion defining a chamber that is in communication with the fourth passageway. Methods and kits are also disclosed.

### TECHNICAL FIELD

The present disclosure generally relates to medical devices, systems and methods that include an inflatable member in biomedical and other medical and non-medical applications, and in particular to apparatuses, systems, methods and kits for preventing over inflation of an inflatable member.

### BACKGROUND

Various types of inflatable members are used during medical procedures to expand an internal cavity of a patient in order to perform a medical procedure. One type of inflatable member is the balloon catheter. In general, balloon catheters can exist in a deflated state and an inflated state; intermediate states are also available. In use, the balloon catheter in its deflated state is inserted into a patient's anatomy. After positioning within the patient, the balloon catheter is inflated via any of various means using various inflation media, for example, using a syringe to inject a liquid mass into the balloon or using an inflation bulb to provide air, liquid or gas into the balloon. Some systems utilize a pressure gauge to monitor the pressure to prevent over pressurization of the balloon.

In some medical procedures an imaging device is used to image a patient's anatomy or structures within the patient's anatomy. In order to capture clear images of the patient's anatomy or structures within the patient's anatomy, the imaging device can be positioned within a balloon catheter that can be inserted into the patient. The balloon is then inflated to provide clear access to the imaging device of the system. In these balloon catheter systems, the balloon catheter and most components connected thereto require disposal due to being in contact with the patient.

In some instances, if an operator is not properly monitoring the pressure gauge, the balloon may be inflated to an over-inflated or over-pressurized state. This over pressurization of the balloon can cause damage to or even rupturing of the balloon, or even worse can cause damage to surrounding tissue. Also, if the balloon is under-inflated, the imaging device may not be able to properly capture and image the patient's anatomy or structures within the patient's anatomy. This disclosure describes an improvement over these prior art technologies.

### SUMMARY

Accordingly, in one embodiment, in accordance with the principles of the present disclosure, an inflation apparatus with pressure relief is provided comprising a housing comprising an interior portion including an opening. A connector is positioned within the interior portion. The connector comprises a first portion defining a first passageway and a second portion defining a second passageway. The second portion includes a pressure relief valve. A shaft defines a third passageway. The shaft extends through the first passageway. An inflatable member defines a fourth passageway. The inflatable member comprises a first end coupled to the connector and a second end positioned external to the interior portion defining a chamber that is in communication with the fourth passageway.

In one embodiment, in accordance with the principles of the present disclosure, an inflation apparatus with pressure relief includes a housing comprising an interior portion including an opening. A connector is positioned in the interior portion adjacent the opening. The connector comprises a first portion defining a first passageway and a second portion defining a second passageway. The second portion includes a pressure relief valve positioned between an inlet and the first portion. The pressure relief valve is positioned within the interior portion and the inlet is positioned external to the interior portion such that the inlet may be coupled to a material source, such as, for example, an air supply or a saline supply present in a hospital room or doctor's office. A first shaft defines a third passageway. The first shaft is disposed in the first passageway and comprises a first end and a second end positioned external to the interior portion. An inflatable member comprises a first end coupled to the first portion and defines a fourth passageway. A second end of the inflatable member is positioned external to the interior portion. The second end of the inflatable member defines a chamber that is in communication with the fourth passageway. The chamber is movable from a collapsed orientation to an expanded orientation by inserting a material into the inlet, through the first, second and fourth passageways and into the chamber. The pressure relief valve is configured to release when pressure within the inflatable member reaches a selected threshold.

In one embodiment, in accordance with the principles of the present disclosure, an inflation apparatus with pressure relief includes a housing extending along a longitudinal axis between a first end and a second end. The housing comprises an inner surface defining an interior portion. The interior portion comprises an opening extending parallel to the longitudinal axis positioned adjacent the first end. An optical coherence tomography (OCT) imaging system is disposed in the interior portion adjacent the second end. A connector is disposed in the interior portion adjacent the opening such that the connector is spaced apart from the imaging system. The connector comprises a first portion extending parallel to the longitudinal axis. The first portion of the connector comprises an inner surface defining a first passageway and a second portion extending perpendicular to the longitudinal axis. The second portion comprises an inner surface defining a second passageway that is in communication with the first passageway. The second portion includes a pressure relief valve positioned within the interior portion between an inlet and the first portion. The inlet is positioned external to the interior portion. The pressure relief valve and the inlet are in communication with the second passageway. The inlet is configured to engage an external material source selected from the group consisting of a gas supply and a liquid supply. The connector further comprises a shutoff valve in communication with the second passageway positioned between the inlet and the pressure relief valve. A first shaft comprises a first end coupled to the imaging system and a second end positioned external to the interior portion. The first shaft further comprises an intermediate portion disposed in the first passageway and an inner surface defining a third passageway. An inflatable member comprises a first end coupled to the first portion of the connector and a second end positioned external to the interior portion. The first end of the inflatable member comprises an inner surface defining a fourth passageway that is in communication with the first passageway and is spaced apart from the third passageway. The second end of the inflatable member comprises an inner surface defining a chamber that is in communication with the fourth passageway. The chamber has the second end of the first shaft positioned therein. An inner member is disposed in the third passageway and comprises a fiber optic cable configured to transmit light energy. The inner member comprises a first end coupled to the imaging system and a second end disposed in the chamber. The second end of the inner member includes an imaging device, such as, for example, a camera or an OCT imaging device that communicates with the imaging system via the fiber optic cable. The chamber is movable from a collapsed orientation to an expanded orientation by inserting a material into the inlet, through the second, first and fourth passageways and into the chamber. The pressure relief valve is configured to release when pressure within the inflatable member reaches a selected threshold.

In one embodiment, in accordance with the principles of the present disclosure, an inflation kit with pressure relief includes more than one air supply for supplying air through a pathway to an inflatable member; a valve connected in the pathway to control the flow of the inflatable member; a pressure gauge connected in the pathway for monitoring the pressure of the inflatable member; and a pressure relief valve connected in the pathway for venting the pressure at a preset pressure.

In one embodiment, in accordance with the principles of the present disclosure, a method for testing an inflation kit includes receiving an inflation kit; attaching a test valve to the pathway configured to seal the pathway; closing test valve to seal the pathway; opening the valve to increase pressure in the pathway; monitoring a pressure gauge; closing the valve upon reaching a preset pressure; determining if the pressure is maintained for a preset period of time; after the preset period of time, opening the valve to again increase the pressure in the pathway; monitoring the pressure gauge; determining if the relief valve opens; identifying on the pressure gauge the pressure at which the relief valve opens.

### BRIEF DESCRIPTION OF THE DRAWINGS

The present disclosure will become more readily apparent from the specific description accompanied by the following drawings, in which:

FIG. 1 is a side, cross sectional view of one embodiment of an inflation system in accordance with the principles of the present disclosure;

FIG. 2 is a breakaway, side, cross sectional view of components of the system shown in FIG. 1;

FIG. 3 is a cross sectional view of components of the system shown in FIG. 1 taken along lines A-A in FIG. 2;

FIG. 4 is a cross sectional view of the system shown in FIG. 1 taken along lines B-B in FIG. 1;

FIG. 5 is a side, cross sectional view of one embodiment of an inflation system in accordance with the principles of the present disclosure;

FIG. 6 is a side, cross sectional view of one embodiment of an inflation system in accordance with the principles of the present disclosure;

FIG. 7 is a cross sectional view of components of the system shown in FIG. 6 taken along lines C-C in FIG. 6; and

FIG. 8 is a cross sectional view of components of the system shown in FIG. 6 taken along lines D-D in FIG. 6.

Like reference numerals indicate similar parts throughout the figures.

### DETAILED DESCRIPTION

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure.

The present disclosure is described herein in connection with an imaging system. It is understood that the present disclosure is applicable to any systems that include an inflatable member, the pressure of which is to be monitored and controlled.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “superior” and “inferior” are relative and used only in the context to the other, and are not necessarily “upper” and “lower”.

Reference will now be made in detail to the exemplary embodiments of the present disclosure, which are illustrated in the accompanying figures.

Inflation system 10 includes a housing 12 extending along a longitudinal axis E between a first end 14 and a second end 16. Housing 12 comprises an inner surface 18 defining an interior portion 20. Interior portion 20 comprises a circular first opening 22 extending parallel to longitudinal axis E positioned adjacent first end 14 and a second circular opening 22a extending parallel to longitudinal axis E positioned adjacent second end 16. In some embodiments, opening 22 and/or opening 22a is coaxial with longitudinal axis E. In some embodiments, first opening 22 and/or second opening 22a is variously shaped and configured, such as, for example, oval, oblong, triangular, square, polygonal, irregular, uniform, non-uniform, offset, staggered, undulating, arcuate, variable and/or tapered, depending upon the requirements of a particular application. In some embodiments, first opening 22 and/or second opening 22a may be disposed at alternate orientations relative to longitudinal axis E, such as, for example, transverse, perpendicular and/or other angular orientations such as acute or obtuse, co-axial and/or may be offset or staggered, depending upon the requirements of a particular application.

In some embodiments, housing 12 is substantially rectangular. However, it is envisioned that housing 12 can have various shape configurations, such as, for example, oval, oblong, polygonal, irregular, uniform, non-uniform, variable and/or tapered, depending upon the requirements of a particular application. In some embodiments, housing 12 is configured to be hand-held and includes an ergonomic configuration and/or gripping features, such as, for example, one or more grooves to facilitate gripping by a medical practitioner, for example. In some embodiments, housing 12 is configured to engage an instrument configured to maintain housing 12 in a selected position relative to the anatomy of a patient and includes engaging features, such as, for example, one or more apertures configured to engage the instrument to fix housing 12 in position relative to the anatomy of a patient.

In some embodiments, housing 12 comprises a metal material, such as, for example, aluminum or stainless steel. In some embodiments, housing 12 comprises a plastic, a polymer and/or composites. In some embodiments, housing 12 is monolithic. In some embodiments, housing 12 includes a first part 24 that defines a first half of interior portion 20 and a second part 26 that defines a second half of interior portion 20, as shown in FIG. 4. In some embodiments, first part 24 is connected to second part 26 by hinge 28, such as, for example, a living hinge in order to allow first part 24 to pivot relative to second part 26, and vice versa. Pivoting first part 24 relative to second part 26 provides access to interior portion 20. In some embodiments, hinge 28 is a barrel hinge, a pivot hinge, a butt/mortise hinge, a case hinge, a continuous hinge, a concealed hinge, a butterfly hinge, a flag hinge, a strap hinge, an H hinge, an HL hinge, a counterflap hinge, a flush hinge, a coach hinge, a rising butt hinge, a double action spring hinge, a tee hinge, a friction hinge, a cranked hinge, a lift-off hinge, a self-closing hinge, or a butt hinge. In some embodiments, first part 24 includes at least two apertures that are aligned with at least two apertures in second part 26. A fastener is inserted into each of the aligned apertures to fix first part 24 with second part 26.

In some embodiments, one of first part 24 and second part 26 includes a first locking element 30 and the other of first part 24 and second part 26 includes a second locking element 32 configured to engage first locking element 30 to fix first part 24 relative to second part 26. In some embodiments, first locking element 30 is a post and second locking element 32 is a hook. In some embodiments, first locking element 30 is a surface and second locking element 32 is a latch. In some embodiments, first locking element 30 is an aperture and second locking element 32 is a latch. In some embodiments, first part 24 is fixed relative to second part 26 by frictional engagement, threaded engagement, mutual grooves, screws, adhesive, nails, barbs and/or raised element, depending upon the requirements of a particular application.

An imaging system 34, such as, for example, an optical coherence tomography (OCT) visualization system is disposed in interior portion 20 adjacent second end 16. In some embodiments, imaging system 34 is fixed relative to housing 12. In some embodiments, imaging system 34 is positioned adjacent first end 14 of housing 12. In some embodiments, imaging system 34 is positioned equidistant between first end 14 of housing 12 and second end 16 of housing 12. In some embodiments, imaging system 34 is coaxial with longitudinal axis E. In some embodiments, imaging system 34 is offset from longitudinal axis E.

A connector 36 is disposed in interior portion 20 adjacent first opening 22 such that connector 36 is spaced apart from imaging system 34. Connector 36 comprises a first portion 38 extending parallel to longitudinal axis E and a second portion 40 extending perpendicular to longitudinal axis E and first portion 38. First portion 38 comprises an inner surface defining a first passageway 42 having a cylindrical cross sectional configuration. Second portion 40 comprises an inner surface defining a second passageway 44 having a cylindrical cross sectional configuration that is in communication with first passageway 42. In some embodiments, first portion 38 and/or second portion 40 may be disposed at alternate orientations, relative to longitudinal axis E, such as, for example, parallel, transverse, perpendicular and/or other angular orientations such as acute or obtuse, co-axial and/or may be offset or staggered, depending upon the requirements of a particular application. In some embodiments, first passageway 42 and/or second passageway 44 may have various cross section configurations, such as, for example, oval, oblong, triangular, rectangular, square, polygonal, irregular, uniform, non-uniform, variable, tubular and/or tapered, depending upon the requirements of a particular application. In some embodiments, connector 36 is fixed relative to housing 12.

Second portion 40 includes a pressure relief valve 50 positioned within interior portion 20 between an inlet 52 and first portion 38. Pressure relief valve 50 is in communication with second passageway 44 and is configured to prevent over-pressuring and/or over-inflating an inflatable member 56. In particular, pressure relief valve 50 is movable between a closed configuration in which pressure within inflatable member 56 is maintained and an open configuration in which pressure within inflatable member 56 escapes through an opening in pressure relief valve 50. Pressure relief valve 50 moves from the closed configuration to the open configuration when pressure within inflatable member 56 reaches a selected threshold pressure. In some embodiments, pressure relief valve 50 includes at least one sensor, such as, for example, a pressure sensor in communication with inflatable member 56 that transmits a signal to pressure relief valve 50 when pressure within inflatable member 56 reaches or exceeds the selected threshold pressure. Upon receiving the signal, pressure relief valve 50 moves from the closed configuration to the open configuration. In some embodiments, a component distinct from pressure relief valve 50, such as, for example, a pressure sensor 85 or a pressure sensor disposed adjacent to inflatable member 56 or within inflatable member 56 sends a signal to pressure relief valve 50 when pressure within inflatable member 56 reaches or exceeds the selected threshold pressure. shutoff valve

In some embodiments, pressure relief valve 50 extends parallel to longitudinal axis E. In some embodiments, pressure relief valve 50 is positioned closer to inlet 52 than first portion 38. In some embodiments, pressure relief valve 50 is positioned closer to first portion 38 than inlet 52. In some embodiments, pressure relief valve 50 is positioned equidistant between inlet 52 and first portion 38. In some embodiments, pressure relief valve 50 may be disposed at alternate orientations relative to longitudinal axis E, such as, for example, transverse, perpendicular and/or other angular orientations such as acute or obtuse, co-axial and/or may be offset or staggered, depending upon the requirements of a particular application. In some embodiments, pressure relief valve 50 is configured to be pre-set to release pressure when pressure within inflatable member 56 reaches a selected threshold pressure, such as, for example, 35 psi. In some embodiments, the selected threshold pressure may be adjusted to accommodate different types of inflatable members, such as, for example, inflatable members made from different materials. In some embodiments, the selected threshold pressure may be adjusted based upon the intended use.

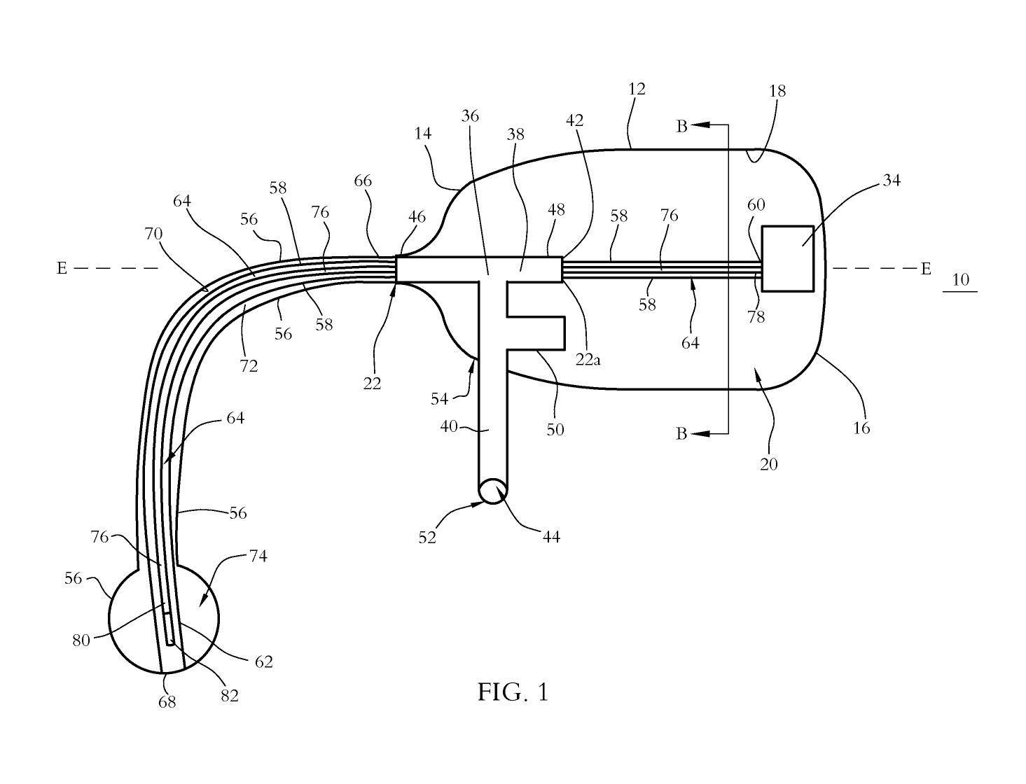

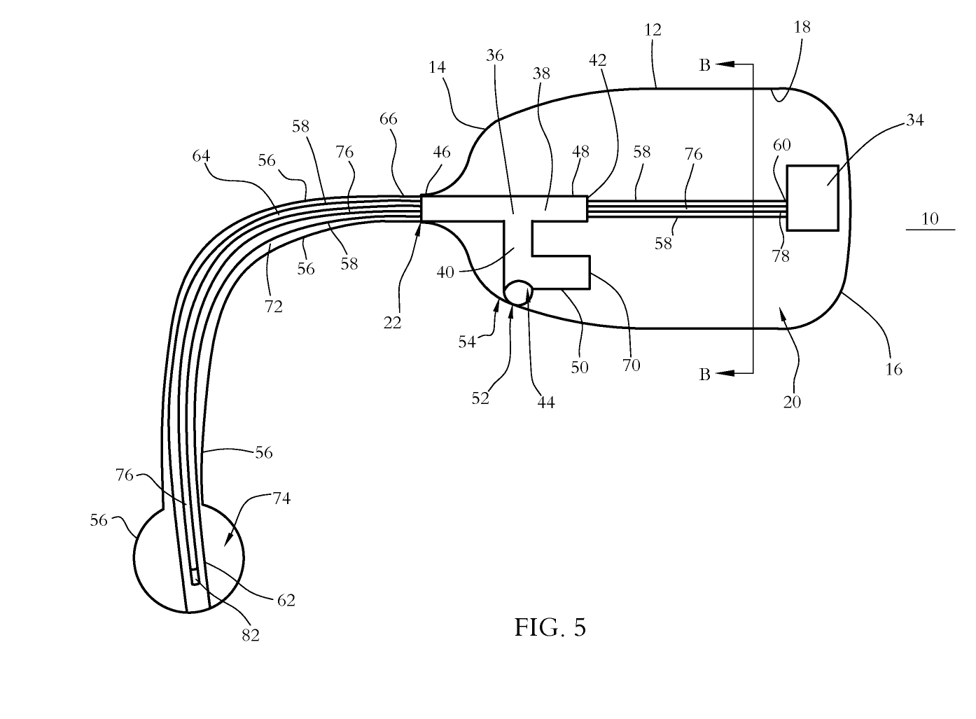

Inlet 52 defines a circular opening that is in communication with second passageway 44. Inlet 52 extends through a circular second opening 54 in housing 12 such that inlet 52 is positioned external to interior portion 20, as shown in FIG. 1. In some embodiments, inlet 52 is positioned within interior portion 20 of housing 12 adjacent second opening 54 such that inlet 52 is accessible from outside interior portion 20, as shown in FIG. 5. Inlet 52 is configured to engage an external material source, such as, for example, a gas supply or a liquid supply already present in a hospital room or doctor's office. It is envisioned that the external material source may include mechanical, electromechanical or pressurized air or liquid supplies, such as, for example, an inflation bulb, a syringe, an electric pump or an air tank containing pressurized air or fluid. It is envisioned that the external material source may supply, for example, air, nitrogen, helium, saline, water and/or contrast media. In embodiments having inlet 52 positioned within interior portion 20, it is envisioned that second opening 54 may include a removable cover configured to prevent any material from entering second passageway 44 through inlet 52 when the material source is not coupled to inlet 52. In some embodiments, inlet 52 and/or second opening 54 may have various cross section configurations, such as, for example, oval, oblong, triangular, rectangular, square, polygonal, irregular, uniform, non-uniform, variable, tubular and/or tapered, depending upon the requirements of a particular application.

In some embodiments, inlet 52 includes engaging features configured to engage the external material source. For example, inlet 52 may include threads on an inner surface of inlet 52 and/or on an outer surface of inlet 52 configured to engage threads of a component of the external material source. In some embodiments, inlet 52 may have various surface configurations, such as, for example, smooth and/or surface configurations to enhance fixation, such as, for example, rough, arcuate, undulating, porous, semi-porous, dimpled, polished and/or textured, to facilitate engagement of inlet 52 with a component of the external material source. In some embodiments, inlet 52 can be variously connected with a component of the external material source, such as, for example, frictional engagement, threaded engagement, mutual grooves, screws, adhesive, nails, barbs and/or raised elements.

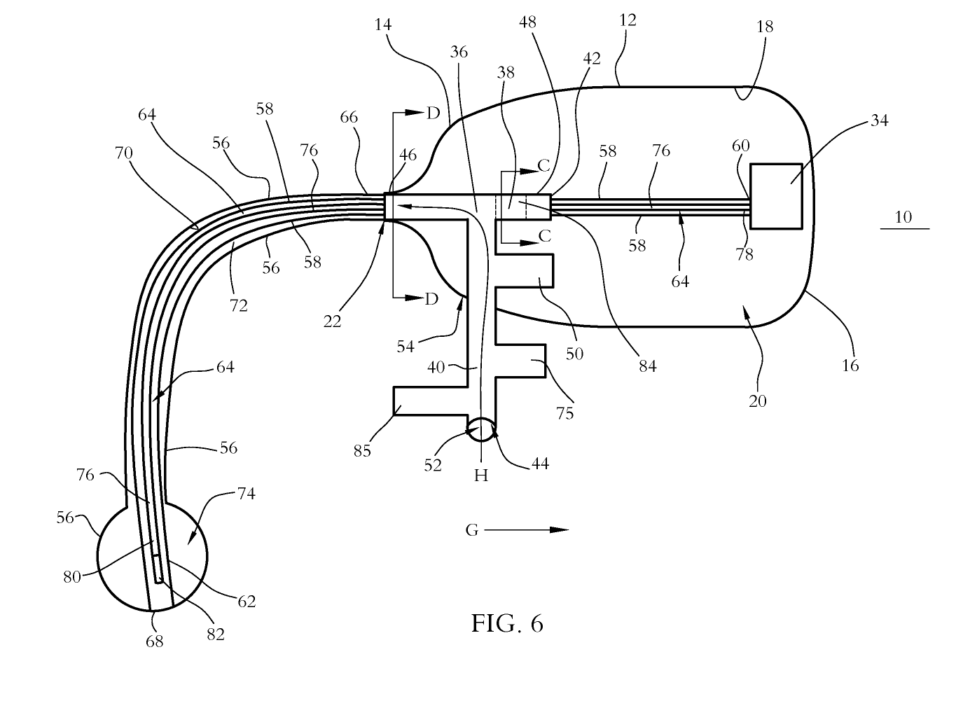

In some embodiments, connector 36 includes a shutoff valve 75 in communication with second passageway 44 positioned between inlet 52 and pressure relief valve 50, as shown in FIG. 6. In some embodiments, shutoff valve 75 is positioned between pressure relief valve 50 and first portion 38. Shutoff valve 75 is movable between a first configuration in which shutoff valve 75 is spaced apart from second passageway 44 when pressure in inflatable member 56 and/or second passageway 44 is below a selected threshold pressure and a second configuration in which shutoff valve 75 blocks second passageway 44 when pressure in inflatable member 56 and/or second passageway 44 reaches a selected threshold pressure to close off second passageway 44 from first passageway 42. In some embodiments, the shutoff valve is in communication with pressure relief valve 50. In some embodiments, shutoff valve 75 automatically closes second passageway 44 off from first passageway 42 when pressure in inflatable member 56 and/or second passageway 44 reaches the selected threshold pressure.

In some embodiments, connector 36 includes a pressure gauge 85 in communication with second passageway 44 positioned between inlet 52 and pressure relief valve 50 configured to gauge the amount of pressure in second passageway 44 and/or inflatable member 56, as shown in FIG. 6. In embodiments that include shutoff valve 75, pressure gauge 85 may be positioned between pressure relief valve 50 and shutoff valve 75 or between inlet 52 and pressure relief valve 50, as shown in FIG. 6, such that pressure gauge 85 is positioned external to interior portion 20. It is envisioned that pressure gauge 85 may be in communication with pressure relief valve 50 and/or shutoff valve 75. In some embodiments, pressure relief valve 50 comprises a pressure gauge. In some embodiments, shutoff valve 75 automatically closes first passageway 42 off from second passageway 44 when pressure in inflatable member 56 and/or second passageway 44 reaches a selected threshold pressure, as detected by pressure gauge 85.

A cylindrical first shaft 58 comprises a first end 60 coupled to imaging system 34 and a second end 62 positioned external to interior portion 20. In some embodiments, shaft 58 is closed and/or sealed at second end 62. First shaft 58 comprises an intermediate portion disposed in first passageway 42. An inner surface of first shaft 58 defines a cylindrical third passageway 64. In some embodiments, first shaft 58 and/or third passageway 64 may have various cross section configurations, such as, for example, oval, oblong, triangular, rectangular, square, polygonal, irregular, uniform, non-uniform, variable, tubular and/or tapered, depending upon the requirements of a particular application. In some embodiments, first shaft 58 comprises a flexible material configured to allow at least a portion of first shaft 58 to bend. In some embodiments, first shaft 58 comprises a rigid material configured to resist bending. In some embodiments, at least one portion of first shaft 58 comprises a flexible material and an adjacent portion of first shaft 58 comprises a rigid material. For example, it is envisioned that a portion of first shaft 58 disposed within interior portion 20 and/or first passageway 42 may comprise a rigid material, while one or more portions of first shaft 58 disposed external to interior portion 20 and/or first passageway comprise a flexible material.

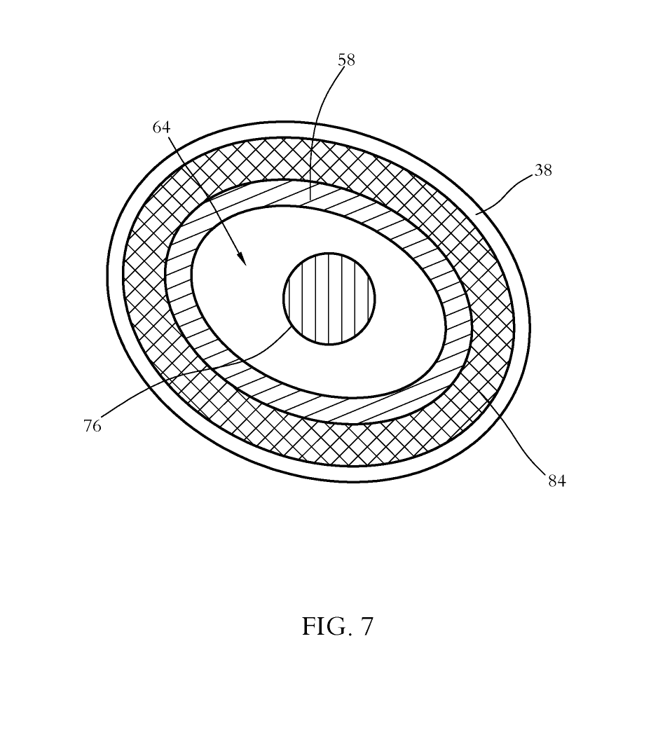

In some embodiments, there is space between an outer surface of shaft 58 and the inner surface of portion 38. In some embodiments, the space between the outer surface of shaft 58 and the inner surface of portion 38 is filled by a blocking member 84, as shown in FIG. 7. That is, blocking member 84 is positioned between the outer surface of shaft 58 and the inner surface of portion 38 such that an inner surface of blocking member 84 engages the outer surface of shaft 58 and an outer surface of blocking member 84 engages the inner surface of portion 38. In some embodiments, the inner surface of blocking member 84 forms an air tight and/or water tight seal with the outer surface of shaft 58 and the outer surface of blocking member 84 forms an air tight and/or water tight seal with the inner surface of portion 38 to prevent liquid and/or gas from flowing in the direction shown by arrow G through portion 38. In some embodiments, blocking member 84 is disposed between an interface between first and second passageways 42, 44 and second end 48 of portion 38 such that liquid and/or gas flows through passageways 42, 44 in the path shown by arrow H. In some embodiments, system 10 comprises a gasket, such as, for example, an O-ring between the inner surface of blocking member 84 and the outer surface of shaft 58 and a gasket between the outer surface of blocking member 84 and the inner surface of portion 38 to provide an air tight and/or water tight seal between the respective components.

Inflatable member 56 comprises a first end 66 coupled to first portion 38 of connector 36 and a second end 68 positioned external to interior portion 20. An inner surface 70 of inflatable member 56 defines a fourth passageway 72 that is in communication with first passageway 42 and is spaced apart from third passageway 64. Inner surface 70 defines a chamber 74 at second end 68 of inflatable member 56 that is in communication with fourth passageway 72. Chamber 74 has second end 62 of shaft 58 positioned therein. In some embodiments, second end 68 of inflatable member 56 is fixed to second end 62 of shaft 58. In some embodiments, second end 68 of inflatable member 56 is spaced apart from second end 62 of shaft 58.

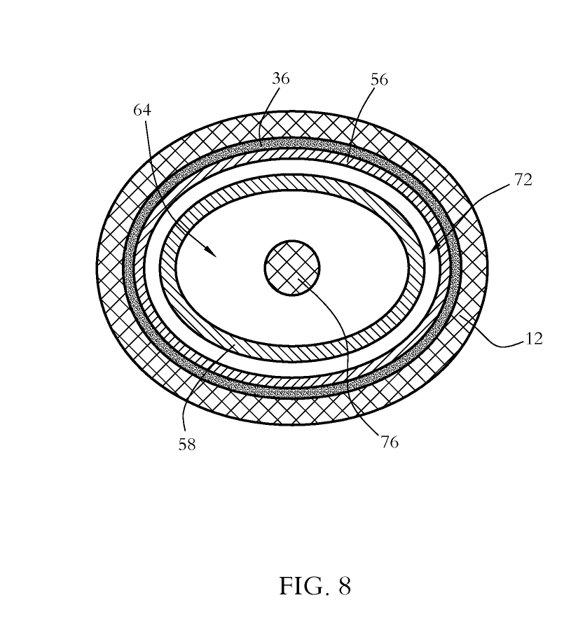

In some embodiments, first end 66 is coupled to first portion 38 of connector 36 such that an outer surface of inflatable member 56 engages the inner surface of first portion 38, as shown in FIG. 8. When the outer surface of inflatable member 56 engages the inner surface of first portion 38, the outer surface of shaft 58 is spaced apart from inner surface 70 by fourth passageway 72, as also shown in FIG. 8. That is, shaft 58 and inflatable member 56 are configured such that fourth passageway 72 remains open and/or unobstructed along the entire length of fourth passageway 72. This configuration allows an inflation and/or filler material to be injected into inlet 52 and move in the direction shown by arrow H such that the material moves from second passageway 44 into first passageway 42. From first passageway 42, the material moves into fourth passageway 72. The material then moves from fourth passageway 72 into chamber 74.

In some embodiments, inflatable member 56 comprises various compliant and/or non-compliant materials, for example, latex and/or polyethylene terephthalate (PET), polyurethane, nylon or polyether block amide. Other materials are also contemplated. In some embodiments, at least a portion of inflatable member 56 comprises a transparent or translucent material to facilitate imaging through inflatable member 56 and/or to allow light from a light source positioned within chamber 74 to travel through inflatable member 56.

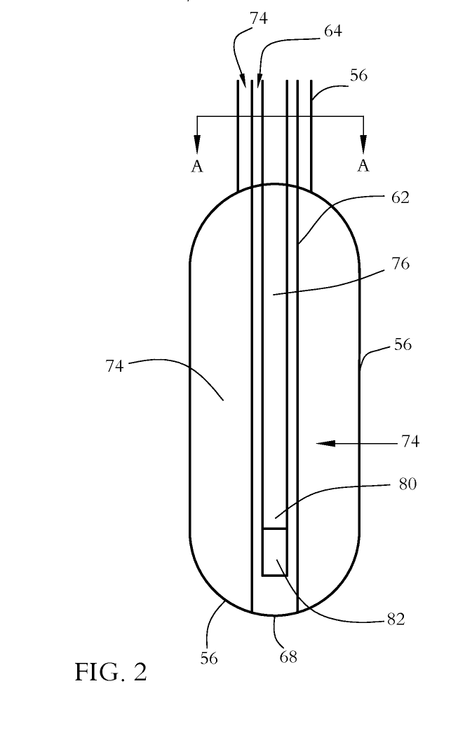

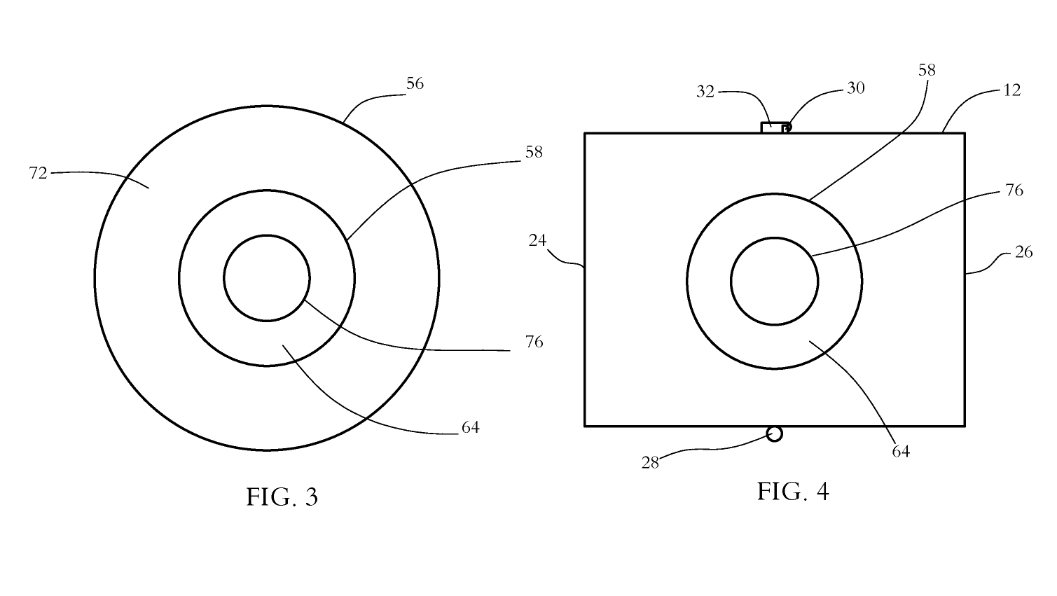

Whichever material is used, inflatable member 56 is configured to transition chamber 74 between a deflated or collapsed orientation and an inflated or expanded orientation; intermediate orientations are also contemplated. Chamber 74 is shown in the expanded orientation in FIGS. 1, 2, 5 and 6. To move chamber 74 from the collapsed orientation to the expanded orientation, an external material source, such as, for example, those described above, is coupled to inlet 52. A material from the external material source is then introduced into second passageway 44 by injection, for example. The material then travels in the direction shown by arrow H such that the material moves through second passageway 44 and into first passageway 42. The material travels from first passageway 42 into fourth passageway 72. The material travels from fourth passageway 72 into chamber 74. As the material is introduced into chamber 74, chamber 74 transitions from the collapsed orientation to the expanded orientation.

Pressure relief valve 50 is configured to prevent an over-pressuring of inflatable member 56 and/or chamber 74. In some embodiments, pressure relief valve 50 is configured to operate over a wide range of pressures. For example, inflatable member 56 may require a pressure of 5 pounds per square inch (psi) as a nominal pressure to move inflatable member 56 from the collapsed orientation to the expanded orientation. Such an inflatable member 56 may have a pressure tolerance rating of +5 psi. As such, pressure relief valve 50 could be configured to release at 8 psi+/−2 to maintain inflatable member 56 within its tolerance ranges to avoid over-pressuring chamber 74 and/or rupturing chamber 74. In some embodiments, inflatable member 56 may require a higher pressure to move inflatable member 56 from the collapsed orientation to the expanded orientation. For example, inflatable member 56 may require a pressure of 30 pounds per square inch (psi) as a nominal pressure to move inflatable member 56 from the collapsed orientation to the expanded orientation. Such an inflatable member 56 may have a pressure tolerance rating of +30 psi. As such, pressure relief valve 50 could be configured to release at 35 psi+/−2 to maintain inflatable member 56 within its tolerance ranges to avoid over-pressuring chamber 74 and/or rupturing chamber 74. Based on the specifications of inflatable member 56, pressure relief valve 50 can be configured to release when the pressure within inflatable member 56 and/or chamber 74 reaches any selected threshold pressure. In some embodiments, pressure release valve 50 is configured to release when the pressure within second passageway 44 and/or first passageway 42 reaches a selected threshold pressure. In some embodiments, at least one pressure gauge is disposed in and/or adjacent chamber 74 to monitor the pressure of chamber 74 as chamber 74 transitions from the collapsed orientation to the expanded orientation.

In some embodiments, system 10 includes an inner member 76 disposed in third passageway 64. In some embodiments, inner member 76 is slideably disposed in third passageway 64. In some embodiments, inner member 76 is rotatably disposed in third passageway 64. In some embodiments, imaging system 34 may include an actuator and/or motor configured to translate inner member 76 axially within third passageway 64 and/or rotate inner member 76 within third passageway 64. Inner member 76 comprises a first end 78 coupled to imaging system 34 and a second end 80 disposed in chamber 74. In some embodiments, inner member 76 comprises a fiber optic cable configured to transmit light energy. In some embodiments, second end 80 of inner member 76 comprises an imaging device 82, such as, for example, an OCT imaging device, a visual light camera, an ultrasound imaging device or other imaging devices. In some embodiments, imaging device 82 includes one or more components commonly found in rotating and/or translating imaging devices. These components can include mirrors, lenses, filters, prisms and combinations thereof; other components are also contemplated.

In operation and use, inflatable member 56 is inserted into the anatomy of a patient, such as, for example, in a cavity in the anatomy of the patient. Once inflatable member 56 is positioned at the desired location, air pressure created by the external material supply is allowed to enter system 10 through inlet 52. In some embodiments, the external material supply is a gas, such as, for example, air. In some embodiments, the external material supply is a liquid, such as, for example, saline. The material travels in the direction shown by arrow H through second passageway 44 and into first passageway 42. The material travels from first passageway 42 into fourth passageway 72. The material travels from fourth passageway 72 into chamber 74. As the material is introduced into chamber 74, chamber 74 transitions from the collapsed orientation to the expanded orientation. During this process, pressure gauge 85 can be checked to monitor the increasing pressure in system 10. In normal operation, this process continues until a selected threshold pressure, e.g. 8 psi or 35 psi, is reached, at which time pressure relief valve 50 may release to prevent over pressurization of inflatable member 56, including chamber 74. That is, pressure relief valve 50 will open at its set pressure, e.g. 8 psi+/−2 or 35 psi+/−2, to prevent damage to system 10 or the patient. In some embodiments, shutoff valve 75 closes off first passageway 42 from second passageway 44, or vice versa, when pressure in inflatable member 56 and/or second passageway 44 reaches the selected threshold pressure, e.g. 8 psi or 35 psi to prevent further pressurization of chamber 74. In some embodiments, pressure relief valve 50 and shutoff valve 75 work simultaneously such that when a selected threshold pressure is detected in chamber 74, relief valve 50 releases simultaneously as shutoff valve 75 closes off first passageway 42 from second passageway 44.

In some embodiments, the selected threshold pressure is determined based on the configuration of inflatable member 56. For example, the selected threshold pressure may be determined based on the material used to form inflatable member 56, based upon the thickness of inflatable member 56 and/or based upon a known pressure limit of inflatable member 56. In some embodiments, the selected threshold pressure is determined based on the intended use. For example, where inflatable member 56 comprises a given material with known characteristics, the selected threshold pressure may be less when inflatable member 56 is used strictly for imaging purposes as opposed to using inflatable member 56 to form a cavity within a patient's anatomy, for example. A medical practitioner may therefore pre-set the selected threshold pressure based upon a variety of factors, thus allowing variability as to when pressure relief valve 50 will release pressure within inflatable member 56. That is, the medical practitioner may select the pre-set threshold pressure at which pressure relief valve 50 will release pressure within inflatable member 56 based on the configuration of inflatable member 56 and/or the intended use of inflatable member 56.

Once chamber 74 is in the expanded configuration, imaging of the patient's anatomy may be accomplished via imaging device 82. Imaging device 82 communicates with imaging system 34 via inner member 76 such that imaging system can generate an image of the patient's anatomy captured by imaging device 82. Due to the configuration of system 10, system 10 can maintain a required pressure within inflatable member 56 to allow the operation of imaging device 82 to translate and/or rotate within the patient.

An inflation kit is also contemplated. The kit can include more than one of components 12-85 described above, wherein the components are variously shaped and/or configured. In some embodiments, the inflation kit includes a plurality of inflatable members, similar to inflatable member 56 having different pre-formed shapes and sizes and/or inflatable members made from different materials such as, for example, compliant materials and non-compliant materials. In some embodiments, the inflation kit includes a plurality of connectors, such as, for example, connectors 38 that are variously shaped and/or configured. In some embodiments, the inflation kit includes each of the connectors 38 shown in FIGS. 1, 5 and 6. In some embodiments, the inflation kit includes more than one air supply for supplying air through a pathway to an inflatable member, such as, for example, inflatable member 56. In some embodiments, the inflation kit includes OCT imaging device, a visual light camera and an ultrasound imaging device. In some embodiments, the inflation kit includes mirrors, lenses, filters and prisms.

In some embodiments, the inflation kit includes a plurality of pressure relief valves, such as, for example, pressure relief valve 50. In some embodiments, the inflation kit includes a plurality of pressure relief valves each configured to release pressure a different pre-set threshold pressure. This configuration allows the inflation kit to include pressure relief valves that are configured to work with specific inflatable members to eliminate the need for a medical practitioner to re-set the threshold pressure at which the pressure relief valve is configured to release pressure, thus saving time and eliminating the possibility of error.

In some embodiments, the inflation kit includes a plurality of shutoff valves, such as, for example, shutoff valve 75. In some embodiments, the inflation kit includes a plurality of shutoff valves each configured to close off one passageway of a connector from another passageway of a connector, when pressure within the connector and/or an inflatable member reaches a different pre-set threshold pressure. This configuration eliminates the need for a medical practitioner to re-set the threshold pressure at which the shutoff valve is configured to close off one passageway of the connector from another passageway of the connector, thus saving time and eliminating the possibility of error. In some embodiments, the inflation kit includes a plurality of pressure gauges, such as, for example, pressure gauge 85. In some embodiments, the inflation kit includes at least one pressure gauge configured to engage a connector and at least one pressure gauge configured for disposal in an inflatable member such that at least two pressure gauges can be used simultaneously.

In some embodiments, the inflation kit includes a test valve used to test the inflation kit. The test valve is attached to inlet 52 with a pressure relief valve, such as, for example, pressure relief valve 50 disconnected from a connector, such as, for example, connector 38 or with the pressure relief valve in an off or inactive state. The test valve is attached to inlet 52 with the test valve in an open configuration such that a material, such as, for example, air or gas may be introduced into second passageway 44 such that the material moves in the direction shown by arrow H through second passageway and into first passageway 42. The material moves through first passageway 42 and fourth passageway 72 and into chamber 74. As the material moves through passageways 42, 44, 72 and into chamber 74, the material increases pressure within passageways 42, 44, 72 and/or chamber 74. The pressure in at least one of passageways 42, 44, 72 and chamber 74 is monitored using a pressure gauge. The test valve closes once the pressure in at least one of passageways 42, 44, 72 and chamber 74 reaches a preset pressure. A user determines if the pressure is maintained for a preset period of time after the test valve closes. After the preset period of time, the test valve is reopened to allow pressure in at least one of passageways 42, 44, 72 and chamber 74 to reduce. In some embodiments, the test valve is removed after the preset period of time. The pressure relief valve is connected to the connector and/or moved from the off or inactive state to an on or active state. The material is moved through passageways 42, 44, 72 and into chamber 74, thus increasing pressure in passageways 42, 44, 72 and/or chamber 74. The pressure gauge is monitored to determine if the relief valve opens when pressure in passageways 42, 44, 72 and/or chamber 74 reaches the preset pressure. The user may identify on the pressure gauge the pressure at which the pressure relief valve opens. The pressure at which the pressure relief valve opens may be compared against the preset pressure to determine whether or not the pressure relief valve opens when pressure in passageways 42, 44, 72 and/or chamber 74 reaches the preset pressure.

The present disclosure has been described herein in connection with an imaging system 10 including an OCT imaging device 82 contained within chamber 74 of inflatable member 56. It is understood that the present disclosure is applicable to any systems that include an inflatable member, the pressure of which is to be monitored, with or without imaging devices as disclosed herein. For example, the present disclosure is applicable to systems for performing procedures such as angioplasty. Other applications are contemplated.

Where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims.

What is claimed is:

1. An inflation system with pressure relief, comprising:
a housing comprising an interior portion including an opening;
a connector positioned within the interior portion, the connector comprising a first portion defining a first passageway and a second portion defining a second passageway, the second portion including a pressure relief valve;
a shaft defining a third passageway, the shaft extending through the first passageway; and
an inflatable member defining a fourth passageway, the inflatable member comprising a first end coupled to the connector and a second end positioned external to the interior portion defining a chamber that is in communication with the fourth passageway.

2. An inflation system as recited in claim 1, wherein:
an end of the second passageway defines an inlet positioned external to the interior portion; and
the pressure relief valve is positioned between the inlet and the first passageway.

3. An inflation system as recited in claim 1, wherein the shaft comprises a first end and an opposite second end positioned external to the interior portion.

4. An inflation system as recited in claim 3, wherein the first end of the shaft is coupled to an imaging system that is fixed within the interior portion.

5. An inflation system as recited in claim 4, wherein the imaging system is an optical coherence tomography (OCT) imaging system.

6. An inflation system as recited in claim 1, wherein an imaging device is disposed in the third passageway such that a first end of the imaging device is coupled to an imaging system that is fixed within the interior portion and a second end of the imaging device is positioned within the chamber.

7. An inflation system as recited in claim 6, wherein the imaging device is slideably disposed in the third passageway.

8. An inflation system as recited in claim 6, wherein the imaging device is rotatably disposed in the third passageway.

9. An inflation system as recited in claim 1, wherein at least a portion of the connector comprises a blocking member is positioned between the connector and shaft.

10. An inflation system as recited in claim 1, wherein the chamber is movable from a collapsed orientation to an expanded orientation by inserting a material into the inlet, through the second, first and fourth passageways and into the chamber.

11. An inflation system as recited in claim 1, wherein the pressure release valve is configured to release pressure within the inflatable member when pressure within the inflatable member reaches a selected threshold.

12. An inflation system as recited in claim 1, wherein the connector is positioned between the first end of the inflatable member and the housing such that an inner surface of the connector engages an outer surface of the inflatable member and an outer surface of the connector engages an inner surface of the housing.

13. An inflation system as recited in claim 1, wherein:
an end of the second passageway defines an inlet positioned external to the interior portion; and
a shutoff valve is positioned between the inlet and the pressure relief valve.

14. An inflation system as recited in claim 13, wherein the shutoff valve is movable between a first configuration in which the shutoff valve blocks a pathway from the inlet to the first passageway and a second configuration in which the shutoff valve does not block the pathway.

15. An inflation system as recited in claim 1, wherein:
an end of the second passageway defines an inlet positioned external to the interior portion; and
a pressure gauge is positioned between the inlet and the pressure relief valve.

16. An inflation system as recited in claim 15, wherein the pressure gauge is positioned external to the interior portion.

17. An inflation system as recited in claim 1, wherein the connector is fixed relative to the housing.

18. An inflation system as recited in claim 1, wherein the
housing includes a first part that defines a first half of the interior portion and a second part that defines a second half of the interior portion, the first part being connected to the second part by a hinge.

19. An inflation system with pressure relief, comprising:
a housing comprising an interior portion including an opening;
a connector positioned within the interior portion adjacent the opening, the connector comprising a first portion defining a first passageway and a second portion defining a second passageway, the second portion including a pressure relief valve positioned between an inlet and the first portion, the inlet being positioned external to the interior portion;
a first shaft defining a third passageway, the first shaft being disposed in the first passageway and comprising a first end and a second end positioned external to the interior portion; and
an inflatable member comprising a first end coupled to the first portion and defining a fourth passageway and a second end positioned external to the interior portion defining a chamber that is in communication with the fourth passageway,
wherein the chamber is movable from a collapsed orientation to an expanded orientation by inserting a material into the inlet, through the second, first and fourth passageways and into the chamber and the pressure release valve is configured to release when pressure within the inflatable member reaches a selected threshold.

20. An inflation system with pressure relief, comprising:
a housing extending along a longitudinal axis between a first end and a second end, the housing comprising an inner surface defining an interior portion, the interior portion comprising an opening extending parallel to the longitudinal axis positioned adjacent the first end;
an optical coherence tomography (OCT) imaging system disposed in the interior portion adjacent the second end;
a connector disposed in the interior portion adjacent the opening such that the connector is spaced apart from the imaging system, the connector comprising a first portion extending parallel to the longitudinal axis and comprising an inner surface defining a first passageway and a second portion extending perpendicular to the longitudinal axis and comprising an inner surface defining a second passageway that is in communication with the first passageway, the second portion including a pressure relief valve positioned within the interior portion between an inlet and the first portion, the inlet being positioned external to the interior portion, the pressure relief valve and the inlet being in communication with the second passageway, the inlet being configured to engage an external material source selected from the group consisting of a gas supply and a liquid supply, the connector further comprising a shutoff valve in communication with the second passageway positioned between the inlet and the pressure relief valve;
a first shaft comprising a first end coupled to the imaging system and a second end positioned external to the interior portion, the first shaft comprising an intermediate portion disposed in the first passageway and an inner surface defining a third passageway;
an inflatable member comprising a first end coupled to the first portion of the connector and a second end positioned external to the interior portion, the first end of the inflatable member comprising an inner surface defining a fourth passageway that is in communication with the first passageway and is spaced apart from the third passageway, the second end of the inflatable member comprising an inner surface defining a chamber that is in communication with the fourth passageway, the chamber having the second end of the first shaft positioned therein; and
an inner member disposed in the third passageway comprising a fiber optic cable configured to transmit light energy, the inner member comprising a first end coupled to the imaging system and a second end disposed in the chamber, the second end of the inner member comprising an imaging device configured to capture images of a patient's anatomy,
wherein the chamber is movable from a collapsed orientation to an expanded orientation by inserting a material into the inlet, through the second, first and fourth passageways and into the chamber and the pressure release valve is configured to release when pressure within the inflatable member reaches a selected threshold.
